(19) United States
(12) Patent Application Publication
(10) Pub. No.: US 2026/0301284 A1
(43) Pub. Date: Oct. 1, 2026

(54) METHOD FOR PROCESSING A MULTISPECTRAL IMAGE DATASET

(71) Applicant: Siemens Healthineers AG, Forchheim (DE)

(72) Inventor: Rainer RAUPACH, Heroldsbach (DE)

(73) Assignee: Siemens Healthineers AG, Forchheim (DE)

(21) Appl. No.: 19/632,921

(22) Filed: Mar. 30, 2026

(30) Foreign Application Priority Data
Mar. 31, 2025 (EP) 25167437.0

(51) Int. Cl. G06T 12/30; A61B 6/00

(52) U.S. Cl. CPC G06T 12/30; A61B 6/5282; G06T 2211/424; G06T 2211/448

(57) ABSTRACT

One or more example embodiments relates to a method for artifact correction, the method comprising transforming primary spectral data; reducing noise from the transformed spectral data; determining artifact images based on the noise-reduced transformed spectral data; transforming the artifact images; and correcting the primary spectral data based on the transformed artifact images.

### CROSS-REFERENCE TO RELATED APPLICATION(S)

The present application claims priority under 35 U.S.C. § 119 to European Patent Application No. 25167437.0, filed Mar. 31, 2025, the entire contents of which is incorporated herein by reference.

### FIELD

One or more example embodiments relates to a method for processing an image dataset which is based on multispectral computed tomography imaging.

### RELATED ART

Metals usually cause artifacts in computed tomography images that cover diagnostically relevant image areas. The physical causes of these artifacts are diverse and, depending on the type and shape of the metal, can be dominated by beam hardening, scanning errors at edges, scattered radiation or signal cancellation. For example, metals with small core charge numbers and densities, such as aluminum or titanium, cause artifacts primarily due to beam hardening.

Various other forms of artifacts are also a fundamental problem in imaging, especially ring artifacts for computed tomography (CT). This is because signal errors in a channel translate into ring-type disturbances through CT reconstruction. Like any type of artifact, they compromise the diagnostic quality of CT images.

In spectral CT, the situation is aggravated by the fact that artifacts in the originally recorded spectral information (“primary spectral data”) are not significant, for example because they have a sufficiently small signal-to-noise ratio and are therefore imperceptible, but become visible when spectral results such as monoenergetic or basic material images are derived.

With regard to beam hardening artifacts of metals, various methods for artifact correction are known. In segmentation-based methods, segmentation using a threshold identifies the metal areas and then, based on the local attenuation, a conclusion is drawn about the relevant metal portion, which in turn allows an estimate of the data error by beam hardening. However, this usually requires a hypothesis as to which metal it is, and is therefore not easily applicable to any metals and alloys.

If spectral information is available, for example in a dual-energy or multi-energy CT, then beam hardening artifacts can be reduced by subtracting the spectral channels. For example, a low-energy image is subtracted from a high-energy image, I=(1+w)I_{Hi}−wI_{Lo} in a weighted manner. Since the beam hardening artifacts are usually more pronounced in the low-energy image than in the high-energy image, they compensate for each other with a suitable mixture. However, this procedure only corresponds to the extrapolation of an even higher energy than I_{Hi}, which of course has fewer beam hardening artifacts. As a major disadvantage, all soft tissue contrasts are therefore greatly reduced, so that this solution is only an illusory solution and can therefore also be described as pseudo-beam hardening correction.

DE 10 2016 204 709 A1 describes a method by which beam hardening artifacts can be reduced when spectral information is available. In particular, it describes a decomposition based on a base material system that has as many degrees of freedom as spectra are available, whereby it is assumed that any other material can be represented as a linear combination of the base materials and can thus also be captured in the sense of beam hardening.

EP 4 343 703 A1 can also be regarded as prior art for the description of methods for beam hardening correction. Here, a transformation A is used to look at combined spectral data instead of real existing base materials. In the sense of the description of one or more example embodiments, these can also be regarded as “base materials” (without a real existing counterpart).

It is not always possible to prevent or physically correct data errors, especially with regard to ring artifacts, with sufficient quality. For this reason, artifacts are often removed using heuristic methods, such as in U.S. Pat. No. 6,044,125 A. This method is representative of the principle of detecting artifacts, creating an artifact image that contains only the artifacts, and then subtracting the artifact image, which leads to their reduction.

### SUMMARY

However, if the significance of the artifacts is too low, then such methods work sufficiently for the primary data, since the artifacts were not relevant at all due to their low significance, but do not prevent the artifacts from becoming visible in results derived from the primary data.

Independent of the grammatical term usage, individuals with male, female or other gender identities are included within the term.

### BRIEF DESCRIPTION OF THE DRAWINGS

The invention is explained in more detail below with reference to the attached figures using exemplary embodiments. The representation in the figures is schematic and highly simplified and not necessarily true to scale. It shows:

FIG. 1 a computed tomography system according to one or more example embodiments,

FIG. 2 an image reconstruction facility with a block diagram of a method for image reconstruction according to one or more example embodiments,

FIG. 3 a flow chart of a first iterative method for image reconstruction according to one or more example embodiments,

FIG. 4 a flow chart of a second iterative method for image reconstruction according to one or more example embodiments,

FIG. 5 a representation of the mapping which is applied to the first image value tuple and the second image value tuple according to one or more example embodiments, and

FIG. 6a flow chart of a method for artifact correction according to one or more example embodiments.

### DETAILED DESCRIPTION

One or more example embodiments relates to a computer-implemented method for processing an image dataset which is based on multispectral computed tomography imaging and which has a first image value tuple and a second image value tuple, wherein the first image value tuple is assigned to a first volume element of an area to be imaged, wherein the second image value tuple is assigned to a second volume element of the area to be imaged, the method comprising:
a calculation of a first portion of a base material by applying a map to the first image value tuple, wherein the first portion of the base material is assigned to the first volume element of the area to be imaged, wherein the map at the first image value tuple has a first gradient, wherein the first gradient is non-zero,
a calculation of a second portion of the base material by applying the map to the second image value tuple, wherein the second portion of the base material is assigned to the second volume element of the area to be mapped, wherein the map at the second image value tuple has a second gradient, wherein the second gradient is non-zero and not equal to the first gradient.

One embodiment provides that a contiguous subspace of the image values has the first image value tuple and the second image value tuple, wherein the map at each image value tuple of the contiguous subspace is continuous.

One embodiment provides that, with respect to a measure of an attenuation of X-rays, the attenuation of the X-rays by the second volume element is stronger than the attenuation of the X-rays by the first volume element,
wherein the gradient of the map is steeper at the second image value tuple than at the first image value tuple.

One embodiment provides that the image dataset has a third image value tuple, wherein the third image value tuple is assigned to a third volume element of the area to be mapped, wherein in respect of the measure of the attenuation of the X-rays, the attenuation of the X-rays by the third volume element is stronger than the attenuation of the X-rays by the second volume element,
wherein a gradient of the mapping at the third image value tuple is flatter than at the second image value tuple.

One embodiment provides that the measure for the attenuation of the X-ray radiation concerns a selected spectrum, in particular the spectrum of the first image value of the image value tuples, of the multispectral computed tomography imaging.

One embodiment provides that the measure for the attenuation of the X-rays relates to a linear combination, in particular in the form of a weighted mixture, of image values assigned to different spectra of the multispectral computed tomography imaging.

One embodiment provides that the measure for the attenuation specifies whether the attenuation of the X-rays exceeds and/or does not reach a predetermined threshold value.

One or more example embodiments relates to an image data processing facility (also referred to as an image data processing system) for processing an image dataset, wherein the image data processing facility is configured to carry out a method according to one or more example embodiments.

One or more example embodiments relates to a computer program product, comprising commands which, when executed by a computer, cause it to carry out the steps of the method according to one or more example embodiments.

One or more example embodiments relates to a computer-readable storage medium, comprising commands which, when executed by a computer, cause it to carry out the steps of the method according to one or more example embodiments.

For example, the imaging apparatus may have the image data processing facility. For example, the imaging apparatus can acquire a first raw imaging dataset with a first radiation that has a first spectrum and/or a first characteristic energy. For example, the imaging apparatus can acquire a second raw imaging dataset with a second radiation that has a second spectrum and/or a second characteristic energy. For example, the image dataset can be reconstructed based on the first raw imaging dataset and based on the second raw imaging dataset using the imaging apparatus. For example, the imaging apparatus can be a dual-energy computed tomography device and/or a multi-energy computed tomography device. For example, the imaging apparatus can be a dual-source computed tomography device and/or a multi-source computed tomography device. For example, the imaging apparatus may have several different filters to filter the radiation.

For example, the image dataset can have one pixel. For example, the pixel can have position information and an image value tuple. The position information can concern, for example, the position of a volume element of an area of an object to be mapped.

Capturing the image dataset may involve acquiring the image dataset using an imaging apparatus. Alternatively, capturing the image dataset may involve loading the image dataset from an image database.

The processing of the image dataset can be carried out in particular via an algorithm that is executed on a computer. For example, the algorithm can comprise the image dataset as input parameters. For example, the algorithm can comprise a result image dataset as an initial value.

The provision of the result image dataset may comprise an output of the result image dataset for a user via an output apparatus and/or a storage of the result image dataset in a database.

In particular, the decomposition image dataset may be and/or comprise a base material image. In particular, a value of a pixel of the decomposition image dataset can be determined based on the second image value tuple.

The provision of the decomposition image dataset may comprise a display of the decomposition image dataset for a user via an output apparatus and/or a storage of the decomposition image dataset in a database.

An image value tuple can be assigned to a volume element of an area of an object to be mapped, for instance. An image value tuple can have multiple image values. For example, an image value can be and/or represent an attenuation value, a computed tomography value, an intensity value, a Hounsfield value, a gray value, a concentration, a density or similar.

The image value tuple can be represented in particular in respect of a base component set. In particular, the base component set can be a base material set and/or have a base material set. The base component set can have multiple base components. In particular, the base material set can have a first base material and/or a second base material. For example, each base component of the base component set can be assigned an image value of the image value tuple. In particular, the image value can be understood as a measure of the presence and/or relevance of the base component to which the image value is assigned in the volume element to which the image value tuple is assigned.

For example, a base component can be a base material, a base energy, a base spectrum, a base parameter or combinations thereof.

A base material can be, for example, a contrast agent, especially iodine, tissue, especially soft tissue, blood, water, bone, cartilage, fat and the like. For example, a base material can be a combination and/or a mixed form of several materials. Additional assumptions, in particular boundary conditions, regarding the density of the multiple materials can be taken into account here. For example, an image value assigned to a base material can correspond to a concentration, a percent by volume, a percent by weight, or a density of the base material, for instance.

For example, a base component set may have a contrast agent, especially iodine, as a first base component and a mixed form of blood and bone as a second base component. Accordingly, the image value tuple can have a contrast agent concentration as the first image value and a concentration of the mixed form as the second image value. The concentration of the mixed form, which is also referred to below as the mixed form concentration, can for example be defined in such a way that with a first mixed form concentration, the concentration of a first material of the mixed form is equal to zero and that with a second mixed form concentration, the concentration of a second material of the mixed form is equal to zero. Instead of the mixed form concentration, the concentration of a material of the mixed form can also be used, wherein a given concentration of the other material of the mixed form is taken into account as a constant contribution. Details relating to base materials are known to the person skilled in the art, especially in the context of the two-material decomposition and three-material decomposition.

A base energy can be, for example, the energy of a radiation with which the image value was recorded. A base spectrum can be for instance the spectrum of a radiation with which the image value was recorded.

An image value, which is assigned to a base energy and/or a base spectrum can be for instance an attenuation value, a computed tomography value, an intensity value or suchlike. Different base energies and/or different base spectra can be achieved in computed tomography, for example, by using different radiation sources, different tube voltages and/or different filters. Details relating to base energies or base spectra are known to the person skilled in the art, especially in the context of dual- or multi-energy computed tomography, dual- or multi-source computed tomography, dual- or multi-spectra computed tomography.

Multispectral computed tomography may include, in particular, multi-energy computed tomography, in particular dual-energy computed tomography, or multi-source computed tomography, in particular dual-source computed tomography, or multi-spectra computed tomography, in particular dual-spectra computed tomography, or similar or a combination thereof.

For example, a base parameter can relate to a type of interaction, for example via a photoelectric effect and/or via Compton scattering, of radiation with an object. A base parameter can be, for example, a suitable combination of an electron density, a nuclear charge number, a mass number and/or other physical and/or chemical parameters that affect the volume element. For example, a base component set can have a first base parameter, which relates to the photoelectric effect, as the first base component, and a second base parameter, which relates to the Compton scattering, as a second base component. Accordingly, the first image value can be an attenuation value, which is a measure of the relevance of the photoelectric effect in the volume element, and the second image value can be an attenuation value, which is a measure of the relevance of the Compton scattering in the volume element.

A base component set for a representation of the image value tuple can be selected from a plurality of possible base component sets. In different base component sets, the image values of the image value tuple can be different and/or have different meanings. It is possible to choose and/or transform between the representations of the image value tuple in different base components sets without departing from the scope of the invention specified by the claims.

The disclosed method aims at correcting the metal-related beam hardening artifacts by exploiting spectral information. This is applicable when energy-resolved CT measurements of the same patient/object are available, such as with dual-energy CT (dual source, twin beam, dual layer, kV switching) or with photon-counting CT.

Based on the diagram of DE 10 2016 204 709 A1, a spectral reconstruction including beam hardening correction with a generalized base material decomposition is proposed.

Hereby a method for image reconstruction is disclosed, the method comprising
a recording of M spectral datasets and reconstruction of M first image data for each spectrum, as described for example in DE 10 2016 204 709 A1,
an execution of a material decomposition in M predetermined materials into material portions b₁, . . . , b_{M}, with the following features:
decomposition of the range of values of the possible local attenuation in the image space (=pixel values) into several, K, subareas, e.g. by threshold values of the pixel values in the first spectrum, or a weighted mixture (e.g. linear combination) of all spectra.
application of a decomposition model, individual to each of the K subareas, of the M spectral image datasets into the M selected materials.

Subsequently, there is a vector of the portions of the M base materials per pixel,
a use of the thus determined base material portions of M−1 materials selected therefrom for the correction of the M spectral datasets, as described for example in DE 10 2016 204 709 A1.

The base material decomposition can be realized in the simplest way as an (affine) linear decomposition with the aid of a M×M matrix D^{(k)} dependent on the subarea k=1, . . . , K,

$$( \begin{matrix}{b}_{1} \\ ⋮ \\ {b}_{M}\end{matrix} ) = {D}^{( k )} \cdot ( ( \begin{matrix}{S}_{1} \\ ⋮ \\ {S}_{M}\end{matrix} ) - ( \begin{matrix}{o}_{1}^{( k )} \\ ⋮ \\ {o}_{M}^{( k )}\end{matrix} ) )$$

More complex decompositions, i.e. a general illustration S→b, can of course also be used.

It is also conceivable to have a larger number of spectra, M′, as base materials, M i.e. M′>M. In this case, the system is overdetermined, but material decomposition is still possible, e.g. with transformation matrices D^{(k)} of dimension M×M′.

In this case, the correction of the spectral datasets will generate correspondingly more (M′) corrected spectral datasets in deviation from DE 10 2016 204 709 A1.

The increase in the visibility of artifacts in derived spectral results is mainly related to two properties of their calculation:

If Si were the primary spectral data for the spectra i=1, . . . , N. Spectral results are typically derived from the form R=ΣᵢwᵢSᵢ, wherein weights can be greater than 1, i.e |wᵢ|>1, and Σᵢwᵢ=1 (e.g., monoenergetic images) or Σᵢwᵢ=0 (certain base materials).

Derived spectral results are more statistically optimized compared to the primary spectral data, i.e. noise is reduced. This generally also increases the signal-to-noise ratio of artifacts.

Hereby a method for artifact correction is disclosed, the method comprising:
a transformation of the primary spectral data by an affine linear transformation, for example in the form

$${S}_{j}^{′} = {\sum }_{i} {A}_{ji} {S}_{i} + {o}_{j}$$

and/or with the property that the quadratic matrix A in particular is invertible,
a noise reduction to the transformed spectral data, for instance to the transformed spectral data

$${S}_{j}^{′} ,$$

wherein noise-reduced data, for instance noise-reduced data

$${\hat{S}}_{j}^{′} ,$$

is produced,
a determination of an artifact image

$${E}_{j}^{′}$$

per degree of freedom, j such that

$${\hat{S}}_{j}^{′} - {E}_{j}^{′}$$

each represents a result with reduced artifacts,
a transformation of the artifact images into the configuration space of the primary spectral data,

$${E}_{i} = {\sum }_{i} {( {A}^{- 1} )}_{ij} {E}_{j}^{′} ,$$

a correction of the primary spectral data by subtracting the transformed artifact images, Sⱼ−Eⱼ.

The order of transformation and noise reduction can be reversed. For example, noise reduction can be performed first on the primary data Si and then affine linear transformation.

One or more example embodiments relates to a computer-implemented method for artifact correction, the method comprising:
a transformation of primary spectral data, wherein in particular transformed spectral data is generated,
a noise reduction of the transformed spectral data, wherein in particular noise-reduced transformed spectral data is generated,
a determination of artifact images based on the noise-reduced transformed spectral data,
a transformation of the artifact images, wherein in particular transformed artifact images are generated,
a correction of the primary spectral data based on the transformed artifact images, wherein in particular artifact-corrected spectral data is generated.

In particular, provision can be made for the transformation of the primary spectral data from an energy-related representation space into a material-related representation space takes place such that the noise reduction of the transformed spectral data takes place in the material-related representation space so that the determination of the artifact images takes place in the material-related representation space, that the transformation of the artifact images from the material-related representation space into the energy-related representation space takes place and that the correction of the primary spectral data takes place on the basis of the transformed artifact images in the energy-related representation space.

One embodiment provides that the transformation of the artifact images is inverse to the transformation of the primary spectral data. One embodiment provides that the transformation of the primary spectral data is invertible and/or is affine linear.

A computer-implemented method for artifact correction is herewith disclosed, the method comprising:
a noise reduction of primary spectral data, wherein in particular noise-reduced spectral data is generated,
a transformation of the noise-reduced spectral data, wherein in particular transformed noise-reduced spectral data is generated
a determination of artifact images based on the transformed noise-reduced spectral data,
a transformation of the artifact images, wherein in particular transformed artifact images are generated,
a correction of the primary spectral data based on the transformed artifact images, wherein in particular artifact-corrected spectral data is generated.

In particular, provision can be made for the noise reduction of the transformed spectral data to take place in an energy-related representation space, for the transformation of the noise-reduced spectral data from the energy-related representation space into a material-related representation space to take place, for the determination of the artifact images to take place in the material-related representation space, for the transformation of the artifact images from the material-related representation space into the energy-related representation space to take place and for the correction of the primary spectral data to take place on the basis of the transformed artifact images in the energy-related representation space.

In particular, it may be provided that the energy-related representation space is spanned by at least two energy-related channels, which can be selected, for example, from energy thresholds, X-ray tube voltages or X-ray radiation filter configurations. In particular, it may be provided that the material-related representation space is spanned by at least two material-related basic parameters, which can be selected, for example, from base material portions, electron densities or effective ordinal numbers. The material-related representation space can be spanned, for example, by a photoelectric portion and a Compton portion.

One embodiment provides that the transformation of the artifact images is inverse to the transformation of the noise-reduced spectral data. One embodiment provides that the transformation of the noise-reduced spectral data is invertible and/or is affine linear.

In particular, the primary spectral data may be primary multispectral data and/or based on a multispectral computed tomography imaging. In particular, the transformation of the primary spectral data can be cross-spectral and/or include a cross-spectral linear combination.

In particular, the artifact-corrected spectral data can be provided. The provision of the artifact-corrected spectral data can comprise an output of the artifact-corrected spectral data for a user via an output apparatus and/or a storage of the artifact-corrected spectral data in a database.

A suitable transformation is given, for example, by

$$A = ( \begin{matrix}1 & 0 \\ - 1 & 1\end{matrix} ) for N = 2 , A = ( \begin{matrix}1 & 0 & 0 \\ - 1 & 1 & 0 \\ - 1 & 0 & 1\end{matrix} ) for N = 3 , …$$

In this way, a spectrum of primary data is distinguished (here without restricting the generality the first), advantageously characterized by the fact that it has the lowest noise of all primary spectra. The other spectra are transformed into the respective difference with respect to the first spectrum. This choice is motivated by the fact that differences between spectral channels with high weight often occur in derived spectral results. On account of the offset of the value range can be brought into the typical range of artifact detection.

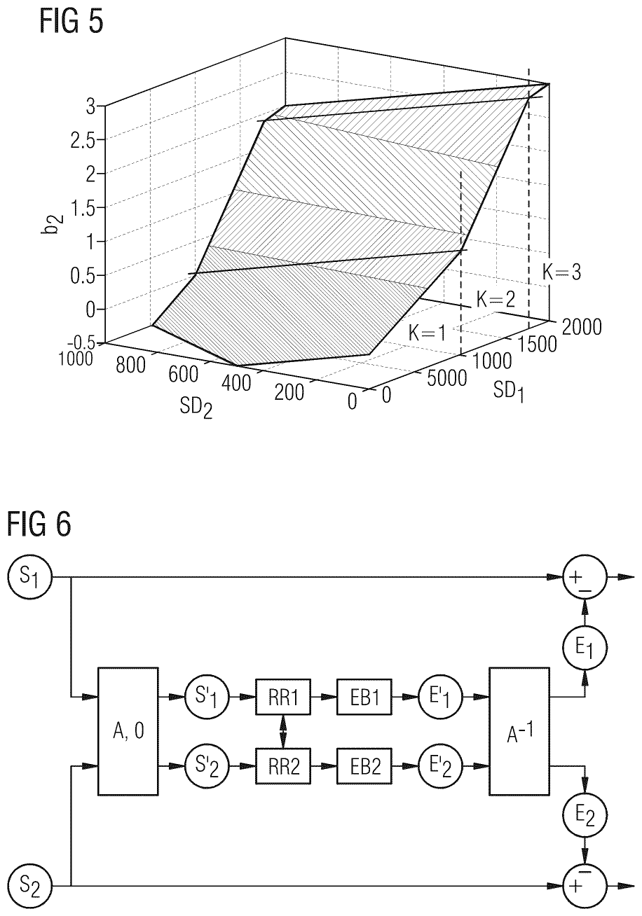

FIG. 6 shows a flowchart of the method for artifact correction for, by way of example, N=2.

In an advantageous way, the noise reduction is morphologically synchronized, as explained for example in DE 10 2019 210 355 A1, since the data depicts the same object/subject, but shows different CT values or contrasts due to the different spectra.

The artifact correction method is not limited to the removal of ring artifacts, but it is particularly relevant for this purpose. The type of acquisition of the primary spectral data is irrelevant. Dual source dual energy, multi-layer detectors, kV switching, or especially thresholds or bins of photon-counting detectors can be used.

The presented method takes into account more materials than are actually available due to the number of degrees of freedom through the spectral information. This is done via the blending between different material systems, such as the local height of the attenuation coefficient. Even if this does not directly indicate the “correct” material system that describes the metal/alloy actually present, the surrogates are much closer to a generic, selected base material system and thus the results are better with regard to beam hardening correction.

This enables artifact reduction by exploiting spectral information from the CT measurement data. The assumption that any other material can be represented as a linear combination of the base materials is generally only approximate, especially if the other material does not occur as a “small perturbation” but in high concentrations. This is the case with metals, which is why the quality of the correction decreases at N (degrees of freedom)=N (spectra).

The proposed method specifically increases the signal-to-noise ratio of the artifacts before detection in such a way that their later visibility is better taken into account in the derived results. This solves the problem that the detection “misses” artifacts because they are not significant enough in the primary data representation.

According to one or more example embodiments, the imaging apparatus is a medical imaging apparatus. According to one or more example embodiments, the imaging apparatus is selected from the group consisting of a C-arm X-ray device, a computed tomography (CT) device, a single-photon emission computed tomography (SPECT) device, a positron emission tomography (PET) device, a magnetic resonance imaging (MRI) device and combinations thereof. In particular, the imaging apparatus may have an X-ray device, an ultrasound device and the like. The imaging apparatus may also be a combination of multiple imaging and/or radiation modalities. A radiation modality can have, for example, an irradiation device for therapeutic irradiation.

According to one or more example embodiments, the image dataset is generated and/or provided via the imaging apparatus. According to one or more example embodiments, the imaging apparatus is embodied to generate and/or provide the image dataset.

One embodiment of the invention provides that the image data processing facility according to one or more example embodiments and/or one or more components of the image data processing facility according to one or more example embodiments are at least partially realized in the form of software on a processor system. In particular, the acquisition module, the processing module, the provision module, the generation module, the selection module and the determination module can each form a component of the image data processing facility according to one or more example embodiments. One embodiment of the invention provides that the image data processing facility according to one or more example embodiments and/or one or more components of the image data processing facility according to one or more example embodiments are at least partially realized in the form of software-assisted hardware, for instance FPGAs, a processor system or suchlike.

Data transfer between components of the image data processing facility can be carried out, for example, via a suitable interface. One embodiment of the invention provides that interfaces for data transfer to and/or from components of the inventive image data processing facility are realized at least in part in the form of software. In particular, the interfaces may have access to suitable storage areas in which data can be appropriately stored, accessed and updated. The interfaces can also be designed as hardware-based interfaces that are controlled by suitable software.

A largely software-based implementation of the image data processing facility according to one or more example embodiments has the advantage that imaging apparatuses and/or computers already used can also be retrofitted by a software update in order to work in the way according to one or more example embodiments. In this respect, the object is also achieved by a corresponding computer program product with a computer program which can be loaded into a storage facility (also referred to as a storage device) of a computer, wherein the computer program carries out the steps of a method according to one or more example embodiments when the computer program is executed on the computer. In addition to the computer program, such a computer program product may include additional software components, e.g. documentation, and/or hardware components, e.g. a hardware key (dongle, etc.) for the use of the software.

A computer-readable medium, for instance a memory stick, a hard disk or any other transportable or permanently installed data carrier on which a computer program is stored, which can be loaded into a storage facility of a computer can be used to transport the computer program and/or to store the computer program on or in a computer, wherein the steps of an inventive method are carried out with the computer program when the computer program is executed on the computer. One embodiment of the invention provides that the imaging apparatus according to one or more example embodiments and/or the image data processing facility according to one or more example embodiments has a computer. The computer can each have a processor system, which has, for example, one microprocessor or several microprocessors working together.

In the context of one or more example embodiments, features described in relation to different embodiments and/or different categories of claims (method, image data processing facility, etc.) can be combined to form further embodiments. In particular, the features, advantages and embodiments described are also to be transferred to the image data processing facility according to one or more example embodiments, the computer program product according to one or more example embodiments and the computer-readable medium according to one or more example embodiments and vice versa. In other words, the claims in question may also be further developed with the features described or claimed in connection with a method. Functional features of a method according to one or more example embodiments can be carried out by appropriately designed components or modules of the image data processing facility according to one or more example embodiments.

The described method and the described image data processing facility are merely embodiments of the invention. One or more example embodiments can be varied by the person skilled in the art, without departing from the scope of the invention insofar as specified by the claims.

The use of the indefinite articles “a” or “an” does not exclude the possibility that the features in question may also be present more than once. The use of the term ‘have’ does not preclude the terms linked by the term ‘have’ from being identical. For example, the medical imaging apparatus has the medical imaging apparatus. It is possible that a module can have several spatially separated sub-modules.

In the context of the present application, the use of ordinal number words (first, second, third, etc.) in the designation of features serves above all to improve the distinctiveness of the features designated by the use of ordinal number words. The absence of a feature which is denoted by a combination of a given ordinal number word and a term does not exclude the possibility that a feature may be present which is denoted by a combination of an ordinal number word following the given ordinal number word and the term.

The expression “based on” can be understood in the context of the present application in particular in the sense of the expression “by the use of”. In particular, a wording according to which a first feature is generated based on a second feature (alternatively: determined, ascertained, etc.) does not exclude the possibility that the first feature can be generated based on a third feature (alternatively: determined, ascertained, etc.).

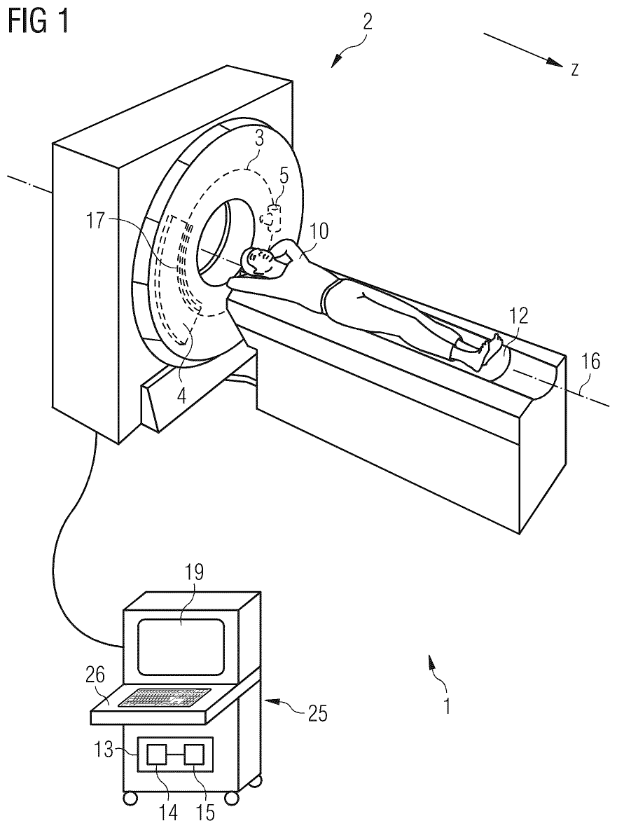

FIG. 1 shows by way of example and roughly schematically a computed tomography system 1, which comprises a user terminal 25 and a computed tomography device 2. The computed tomography system 1 is designed to execute the image reconstruction method. The computed tomography device 2 comprises a patient table 12 for positioning a patient 10 as an examination object, which can be adjusted along a system axis 16. The system axis 16 is also referred to below as the z-axis, which can be adjusted into the measuring field with the patient 10. It also includes a gantry 3 with a source-detector arrangement 4, 5 that can be rotated around the system axis 16. The source-detector arrangement 4, 5 has an X-ray source 5 and a quantum-counting detector 4, which are oriented opposite each other in such a way that during operation an X-ray radiation emanating from the focus of the X-ray source 5 hits the detector 4. The detector 4 is structured into individual pixels 17 for the spatially resolved detection of X-rays, which are arranged into several detector lines. Detectors 4 are currently used, which have a total of 64 or more lines and have a spatial resolution in the submillimeter range. For each projection, the detector 4 generates a set of projection data. The projection data represents the attenuation values of all pixels 17 of an X-ray weakened by the patient 10. Depending on their energy, they are detected in separate bins of the pixels 17 of the detector 4. The portions of the projection data of all projections that were recorded with the same energy in corresponding bins are here a spectral raw dataset SD1, SD2, . . . , SDN. The spectral raw datasets SD1, SD2, . . . , SDN are forwarded to the user terminal 25 with an image reconstruction facility 13 (also referred to as an image data reconstruction system) and are processed into a resulting image via a method for processing an image dataset, which can be displayed e.g. on a display unit 19 and/or which can be stored in a memory and/or sent to other systems. For this purpose, the image reconstruction facility 13 comprises a combination module 14 and an optimization module 15. The user terminal 25 also comprises a keyboard 26 as an input device, with which an operator can set values for parameters in image reconstruction if necessary.

Such a computed tomography device 2 is used as is known for 3D image reconstruction. To acquire an image of a region of interest, projection data from a variety of different projection directions is recorded in an energy-resolved manner in bins as spectral raw datasets SD1, SD2, . . . , SDN by rotating the source-detector arrangement 4, 5. In the case of spiral scanning, for example, during a rotation of the source-detector arrangement 4, 5, a continuous adjustment of the patient table 12 in the direction of the system axis 16 takes place at the same time. In this type of scanning, the X-ray source 5 and the detector 4 thus move on a helical path around the patient 10.

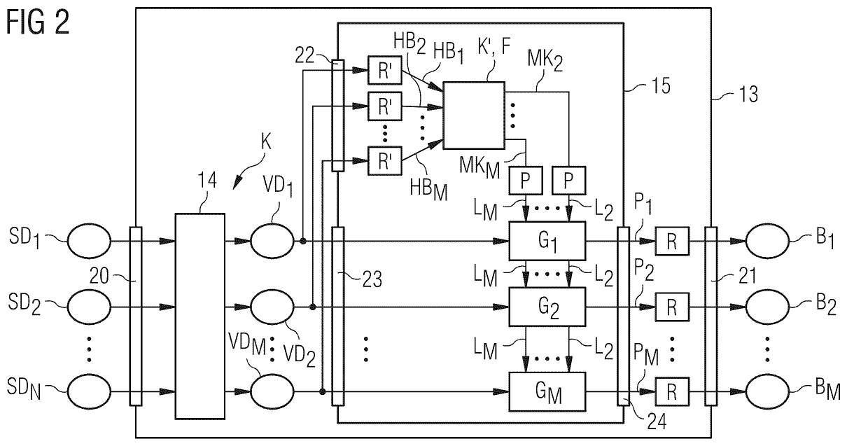

FIG. 2 shows the image reconstruction facility 13 with a block diagram of a method for image reconstruction. The image reconstruction facility 13 has a combination module 14 and an optimization module 15 and an input interface 20 and an output interface 21. The input interface 20 receives a number of N raw spectral datasets SD1, SD2, . . . , SDN acquired by the computed tomography device 2 and forwards them to the combination module 14. The combination module 14 performs a linear combination K of the spectral raw datasets SD1, SD2, . . . , SDN and generates a number of M virtual raw datasets VD1, VD2, . . . , VDM in the process. From the generation of the virtual raw datasets VD1, VD2, . . . , VDM, the indices denote

1, 2, . . . , M virtual spectral channels. They are therefore assigned to the correspondingly combined spectrum and later also to a material.

In the linear combination K, the spectral raw datasets SD1, SD2, . . . , SDN for each virtual raw dataset VD1, VD2, . . . , VDM are multiplied by coefficients, i.e. scalar factors, and then added together so that they have defined average or effective energies at the operator's choice and/or are statistically correlated as weakly as possible. The number M is preferably 3, especially preferably 2, and thus lower than the number N of the spectral raw datasets SD1, SD2, . . . , SDN, since the detector 4 usually resolves the spectrum of X-rays into more energy thresholds than can currently be used sensibly for the subsequent material-dependent optimization of the reconstruction.

The optimization module 15 has a first input interface 22 and a second input interface 23 as well as an output interface 24. The virtual raw datasets VD1, VD2, . . . , VDM are transferred from the combination module 14 to the first input interface 22 for the creation of optimizing mapping functions G1, G2, . . . , GM as well as to the second input interface 23 as arguments for the optimizing mapping functions G1, G2, . . . , GM.

The virtual raw datasets VD1, VD2, . . . , VDM received from the first input interface 22 are each reconstructed to form auxiliary image datasets HB1, HB2, . . . , HBM via an auxiliary filtered rear projection R′. This is followed by another, optional linear combination K′ of the auxiliary image datasets HB1, HB2, HBM to form newly weighted auxiliary image datasets that are assigned to a material and, for example, are again statically correlated as weakly as possible. One of the auxiliary image datasets HB1 is assigned to the material “water”, which is selected (according to step c) of the method described above). From the remaining M−1 auxiliary image datasets HB2, HB3, . . . , HBM, material-specific maps MK2, MK3, . . . , MKM are calculated voxel-wise via a threshold function F. These essentially depict the local concentrations of the respective materials, but can also represent an electron density map or a nuclear charge map.

Subsequently (according to step e) of the above-mentioned method), material line integrals L2, . . . , LM are obtained for each material via a virtual forward projection P under the same geometry as in the acquisition of the original data from the material-specific maps MK2, MK3, . . . , MKM. In the following, M optimizing mapping functions G1, G2, . . . , GM are essentially generated by a linear combination of material line integrals L2, . . . , LM weighted with the absorption coefficients μ2, μ3, . . . , μM. In addition, the first component of the respective mapping function Gj with j=1, . . . , M, which is assigned to the selected material, is formed as function hj of the material line integrals L2, . . . , LM, the absorption coefficient μ1j of the selected material and the virtual raw dataset VD1, VD2, . . . , VDM assigned to the respective virtual spectral channel j=1, . . . , M as an argument as follows:

$$Gj ( VDj , L 2 , … , LM ) = hj ( VDj ; L 2 , … , LM ) μ 1 j + L 2 μ 2 j + L 3 μ 3 j + …$$

With the optimizing mapping functions G1, G2, . . . , GM (according to step f) of the above-mentioned method), optimized synthetic projection data P1, P2, . . . , PM is generated from the virtual raw datasets VD1, VD2, . . . , VDM as arguments. These leave the optimization module 15 via the output interface 24.

In the next step (corresponding to step g) of the above-mentioned method), the synthetic projection data P1, P2, . . . , PM is reconstructed to form image datasets B1, B2, . . . , BM via filtered rear projection R. These are transmitted to the output interface 21 and can then be displayed, for example, on the display unit 19 of the user terminal 25 of the computed tomography system 1 or also transmitted to any other display units and/or storage facilities.

All data of the described method, in other words of the spectral raw data SD1, SD2, . . . , SDN to the image data B1, B2, . . . , BM and in particular also the mapping function Gj generally have a dependency on parameters of the beam. Parameters of the beam are, for example, projection angle, channel number, detector line and the like. This relationship exists, for example, because the X-ray spectrum changes due to a shape filter as a function of the distance from the center of rotation. Regardless of the parameters of the beam, variables such as the absorption coefficients μ naturally remain.

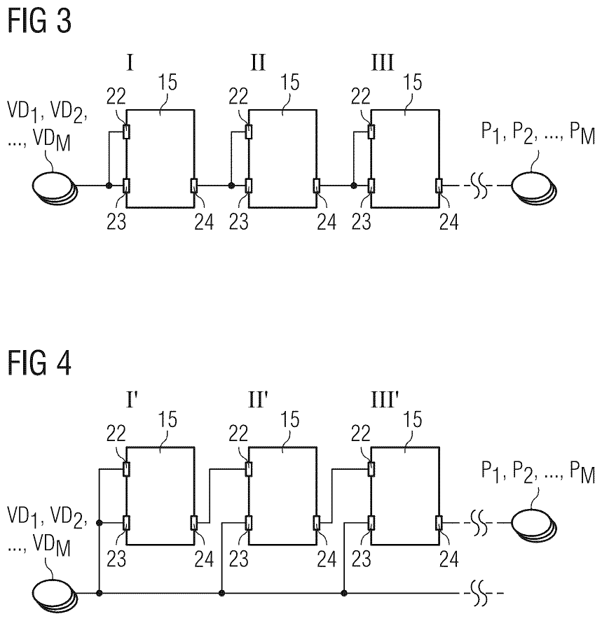

FIG. 3 shows by way of example a schematic representation of a first variant of an iterative method for image reconstruction. The virtual raw datasets VD1, VD2, . . . , VDM have already been combined in a preparatory step using the combination module 14 from the spectral raw datasets SD1, SD2, . . . , SDN (not shown here). In any number of optimization runs I, II, III, . . . , optimized synthetic projection datasets P1, P2, . . . , PM are generated therefrom. For this purpose, an optimization module, which is particularly preferably designed as a software module, is run through several times. A corresponding number of optimization modules 15 in series is clearly shown here as an alternative version. The first optimization module 15 receives the virtual raw datasets VD1, VD2, . . . , VDM at both input interfaces 22, 23. It then calculates synthetic projection datasets P1, P2, . . . , PM and forwards them via its output interface 24 to both input interfaces 22, 23 of the optimization module 15 located behind it. This process is repeated for any number of passes. Finally, i.e. as soon as the desired degree of optimization has been achieved, the optimized synthetic projection datasets P1, P2, . . . , PM are output and subsequently reconstructed into image datasets B1, B2, . . . , BM as already described (not shown here).

FIG. 4 shows by way of example and schematically a second variant of an iterative method for image reconstruction. The FIG. 4 is similar to FIG. 3. The difference is that only the first input interface 22 of the respective optimization module 15 contains the optimized synthetic projection datasets P1, P2, . . . , PM from the previous optimization run I′, II′, III′, . . . as virtual raw data records VD1, VD2, . . . , VDM. The second input interface 23, on the other hand, always receives, i.e. in each run, the original virtual raw datasets VD1, VD2, . . . , VDM.

The described solution enables a flexible use of the multispectral information to reduce beam hardening artifacts or to calculate monochromatic images or base material images. It thus combines the advantages of image- and raw data-based methods and avoids the respective disadvantages.

FIG. 5 shows a representation of the mapping applied to the first image value tuple and the second image value tuple, and the division into three subareas based on a degree of the attenuation. The mapping is contiguous at the subarea boundaries and goes into saturation at K=3.

FIG. 6 shows a flow chart of a method for artifact correction. In steps RR1 and RR2, noise reduction is carried out. In steps EB1 and EB2, an artifact image is ascertained.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SOC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable medium storage including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for artifact correction, the method comprising:
transforming primary spectral data;
reducing noise from the transformed spectral data;
determining artifact images based on the noise-reduced transformed spectral data;
transforming the artifact images; and
correcting the primary spectral data based on the transformed artifact images.

2. The method of claim 1, wherein the transforming the artifact images is inverse to the transforming the primary spectral data.

3. The method of claim 1, wherein the transforming the primary spectral data is at least one of invertible or affine linear.

4. The method of claim 1, wherein the reducing is morphologically synchronized.

5. The method of claim 1, wherein the spectral data is based on a multispectral computed tomography imaging.

6. A computer-implemented method for processing an image dataset which is based on multispectral computed tomography imaging, the image dataset having a first image value tuple and a second image value tuple, wherein the first image value tuple is assigned to a first volume element of an area to be imaged, wherein the second image value tuple is assigned to a second volume element of the area to be imaged, the method comprising:
calculating a first portion of a base material by applying a mapping to the first image value tuple, wherein the first portion of the base material is assigned to the first volume element of the area to be imaged, wherein the mapping to the first image value tuple has a first gradient and the first gradient is non-zero; and
calculating a second portion of the base material by applying the mapping to the second image value tuple, wherein the second portion of the base material is assigned to the second volume element of the area to be imaged, wherein the mapping at the second image value tuple has a second gradient, the second gradient is non-zero and not equal to the first gradient.

7. The method of claim 6, wherein
a contiguous subspace of the image values has the first image value tuple and the second image value tuple, and
the mapping is continuous at each image value tuple of the contiguous subspace.

8. The method of claim 6, wherein
with respect to a measure of an attenuation of X-rays, an attenuation of the X-rays by the second volume element is stronger than an attenuation of the X-rays by the first volume element, and
the gradient of the mapping is steeper at the second image value tuple than at the first image value tuple.

9. The method of claim 8, wherein
the image dataset has a third image value tuple, wherein the third image value tuple is assigned to a third volume element of the area to be imaged,
an attenuation of the X-rays by the third volume element is stronger than the attenuation of the X-rays by the second volume element, and
a gradient of a mapping at the third image value tuple is flatter than at the second image value tuple.

10. The method of claim 8, wherein the measure of the attenuation of the X-rays concerns a selected spectrum.

11. The method of claim 8, wherein the measure of the attenuation of the X-rays relates to a linear combination.

12. The method of claim 8, wherein the measure of the attenuation specifies whether the attenuation of the X-rays at least one of exceed or does not reach a predetermined threshold value.

13. An image data processing system comprising:
a memory storing instructions; and
processing circuitry configured to execute the instructions to cause the image data processing system to perform the method of claim 1.

14. A non-transitory computer program product, comprising commands, upon execution by a computer, cause the computer to perform the method of claim 1.

15. A non-transitory computer-readable storage medium, comprising commands, upon execution by a computer, cause the computer to perform the method of claim 1.

16. The method of claim 10, wherein the selected spectrum is a spectrum of the first image value of the image value tuples of the multispectral computed tomography imaging.

17. The method of claim 11, wherein the linear combination is a weighted mixture of different spectra of image values assigned to the multispectral computed tomography imaging.

18. The method of claim 9, wherein the measure of the attenuation of the X-rays concerns a selected spectrum.

19. The method of claim 9, wherein the measure of the attenuation of the X-rays relates to a linear combination.

20. The method of claim 9, wherein the measure of the attenuation specifies whether the attenuation of the X-rays exceeds and/or does not reach a predetermined threshold value.
